(19) United States
(12) Patent Application Publication
(10) Pub. No.: US 2007/0116699 A1
(43) Pub. Date: May 24, 2007

(54) Nattokinase for reducing whole blood viscosity

(75) Inventor: Ralph E. Holsworth, Pagosa Springs, CO (US)

Correspondence Address: FOLEY AND LARDNER LLP;SUITE 500, 3000 K STREET NW, WASHINGTON, DC, 20007, US

(73) Assignee: N-ZymeCeuticals, Inc.

(21) Appl. No.: 11/473,344

(22) Filed: Jun. 23, 2006

Related U.S. Application Data
(60) Provisional application No. 60/693,409, filed on Jun. 24, 2005.

(51) Int. Cl. A61K 38/48

(52) U.S. Cl. 424/94.63

(57) ABSTRACT

Disclosed is a method for reducing whole blood viscosity in a patient in need thereof. The method includes administering a composition that comprises nattokinase. The composition may be administered to any patient that may benefit from a reduction in blood viscosity, such as a patient having or at risk for vascular diseases and conditions.

### CROSS-REFERENCE TO RELATED APPLICATIONS

This application claims the benefit under 35 U.S.C. § 119(e) of U.S. provisional application No. 60/693,409, filed on Jun. 24, 2005, the entire contents of which are incorporated herein by reference.

### BACKGROUND

The invention presented herein relates generally to the field of compositions and methods for reducing whole blood viscosity. In particular, the invention relates to nattokinase compositions useful for reducing whole blood viscosity in a patient in need thereof.

Nattokinase, also called Substilisin NAT (Enzyme Commission Number EC 3.4.21.62 and CAS Registry Number 9014-01-1), is a pro-fibrinolytic enzyme that is present in a vegetable cheese-like food called Natto, which is extremely popular in Japan and has been consumed for over 1,000 years. Natto is prepared by fermenting boiled soybeans with Bacillus spp. (e.g., Bacillus subtilis in particular Bacillus subtilis var. natto). Traditionally, Natto was consumed as a folk remedy to treat various conditions and ailments. Nattokinase may be extracted and purified from Natto.

Nattokinase is a subtilisin-like serine protease that is expressed as a 381 amino acid pro-enzyme which is cleaved to produce a 275 amino acid processed form having a molecular weight of approximately 27.7 kDa. Nattokinase has been shown to possess fibrinolytic activity in vitro and in vivo. Studies have indicated that oral administration of Nattokinase may be beneficial for treating essential hypertension and reducing thrombosis.

Recently, rheological parameters such as elevated whole blood viscosity have been suggested to be risk factors for conditions such as cardiovascular disease. There is a need, therefore, for compositions and methods for treating various hemorheological parameters, such as elevated whole blood viscosity. Although nattokinase's fibrinolytic properties have led to the use of nattokinase to reduce thrombosis, the ability of nattokinase to affect hemorheological parameters and to reduce whole cell viscosity have not heretofore been explored, and methods of reducing whole cell viscosity using nattokinase compositions have not heretofore been proposed. Agents that reduce blood viscosity or prevent high blood viscosity (i.e., “anti-viscogenic agents”) may be useful for treating a variety a conditions associated with vascular dysfunction.

### SUMMARY

The present invention provides compositions and methods for reducing whole blood viscosity.

In accordance with some embodiments, the invention provides a method for reducing whole blood viscosity in a patient in need thereof, comprising administering to the patient a composition that comprises nattokinase.

In accordance with other embodiments, the patient is at risk for or has one or more conditions or diseases selected from cerebral vascular injury (e.g., arising from a stroke), cardiovascular disease and/or injury (e.g., patients exhibiting elevated plasma concentration of lipoprotein(a) (Lp(a)) and having increased risk for atherogenesis), telangiectasia or “spider veins,” chronic venostatis (i.e., “venous stasis”) or varicose veins, essential hypertension, diabetes, conditions associated with pregnancy (e.g., eclampsia, pre-eclampsia, or hyperviscosity associated with pregnancy while residing at a high altitude), hepatic disease and/or injury, renal disease and/or injury, cerebral disease and/or injury, pancreatic disease and/or injury, anemia (e.g., while undergoing erythropoietin therapy), headaches (e.g., migraine headaches to reduce cephalgia), heavy metal poisoning, osteoarthritis, Behcet's disease, Chagas' disease, Cushing syndrome, and Waldenstrom's disease. Patients may include those patients who are undergoing therapy with drugs used to improve blood viscosity (e.g., anti-platelet drugs such as aspirin or blood thinning drugs such as warfarin). Patients may include those patients who have developed resistance to the effects of drugs used to improve blood viscosity (e.g., resistance to the anti-platelet effect of aspirin). The patient may be administered the nattokinase composition as part of preventive therapy, rehabilitative therapy, or both.

In accordance with other embodiments, as a result of the method, the patient's whole blood viscosity is reduced an average of at least about 5% over a shear rate range of 1-1000 s⁻¹ at a hematocrit of about 30-50%. In some embodiments, the patient's whole blood viscosity is reduced an average of at least about 10%, at least about 15%, at least about 20%, at least about 25%, or at least about 30%, over a shear rate range of 1-1000 s⁻¹ at a hematocrit of about 30-50%.

In accordance with other embodiments, as a result of the method, the patient's red blood cell aggregation is reduced. In accordance with another embodiment, as a result of the method, the patient's red blood cell deformability is increased.

In accordance with one embodiment, the nattokinase is administered at a dosage of at least about 2,000 fibrin units per day, at least about 4,000 fibrin units per day, or at least about 6,000 fibrin units per day.

In accordance with other embodiments, the nattokinase comprises a polypeptide having an amino acid sequence of SEQ ID NO:3, or at least 200 contiguous amino acids of SEQ ID NO:3. In accordance with another embodiment, the nattokinase comprises a variant polypeptide having at least about 90% sequence identity to SEQ ID NO:3 (or at least about 95% sequence identity to SEQ ID NO:3). The variant polypeptide preferably has nattokinase activity (e.g., fibrinolytic activity).

In accordance with some embodiments, the nattokinase is prepared from an extract of soy beans that have been fermented with Bacillus subtilis var. natto.

In accordance with some embodiments, the method further includes administering at least one other therapeutic agent. In one embodiment, the method further includes administering at least one anti-coagulant (e.g., aspirin, Coumadin, and mixtures thereof). In another embodiment, the method further includes administering at least one lipid lowering agent (e.g., statin or lipase). In another embodiment, the method further includes administering at least one protease (e.g., bromelain, papain, lumbrokinase, and mixtures thereof). In another embodiment, the method further includes administering at least one angiotensin-converting enzyme inhibitor. In another embodiment, the method further includes administering at least one calcium channel blocker. In another embodiment, the method further includes administering a diuretic.

### BRIEF DESCRIPTION OF THE DRAWINGS

FIG. 1 represents the red blood cell aggregation index in plasma for whole blood treated with nattokinase at various doses.

### DETAILED DESCRIPTION

The present invention provides a method of reducing whole blood viscosity in a patient in need thereof. The method typically includes administering a composition that comprises nattokinase to the patient. The method may be utilized to reduce whole blood viscosity in any patient in need thereof.

Patient Populations

A number of different types of patients may benefit from the method of the present invention. As used herein, a “patient in need thereof” is any patient that would benefit from a treatment regimen in which whole blood viscosity is reduced, including the patients described below.

A “patient in need thereof” may include a patient with a vascular disease or at risk for a vascular disease. For example, a “patient in need thereof” may include a patient with a cardiovascular disease or injury or a patient at risk for developing cardiovascular disease or injury. Patients with cardiovascular disease or injury may benefit from a treatment regimen that results in a decrease in whole blood viscosity. A reduction in whole blood viscosity may benefit these patients by improving circulation and consequently reducing the work load of the heart. A patient with cardiovascular injury may include a patient that is undergoing or that has undergone cardiovascular surgery.

A “patient in need thereof” also may include a patient with essential hypertension or at risk for developing essential hypertension. For example, by reducing whole blood viscosity, blood pressure (i.e., to treat essential hypertension) may be consequently reduced. “Essential hypertension,” as used herein, may include a syndrome without an identified etiology in which the patient has a systolic blood pressure of at least about 170 mm Hg (including a systolic blood pressure of at least about 180 mm Hg, 190 mm Hg, or 200 mm Hg). “Essential hypertension” may also include a syndrome without an identified etiology in which the patient has a diastolic blood pressure of at least about 100 mm Hg (including a diastolic blood pressure of at least about 110 mm Hg, 120 mm Hg, or 130 mm Hg): For such patients, nattokinase may be administered (before or after) or co-administered with an anti-hypertensive agent to prevent or reduce the likelihood of heart attacks, strokes, and/or aneurysms.

A “patient in need thereof” also may include a patient with diabetes or a patient at risk for developing diabetes. As used herein, “diabetes” may include type I diabetes (i.e., juvenile diabetes) and/or type II diabetes (i.e., adult-onset diabetes). For example, diabetics often suffer from poor circulation which may be improved by reducing whole blood viscosity.

A “patient in need thereof” may include a pregnant women who has or who is at risk for developing elevated whole blood viscosity and/or complications associated with elevated whole blood viscosity. For example, a “patient in need thereof” may include a pregnant woman who has or who is at risk for developing intrauterine growth restriction and/or preeclampsia. A pregnant women who resides at or who intends to reside at a high altitude during pregnancy may have or may be at risk for developing elevated whole blood viscosity and complications linked to elevated whole blood viscosity, such as intrauterine growth restriction and/or preeclampsia. Kametas et al., “Pregnancy at high altitude: a hyperviscosity state,” ACTA OBSTET. GYNECOL SCAND. (2004), 83(7):627-33. “High altitude” typically means at least about 3000 meters above sea level, and includes at least about 4000 meters or 5000 meters above sea level.

A “patient in need thereof” also may include a patient who has or who is at risk for developing disease or injury to organs such as, but not limited to, the heart (and/or cardiovascular system), liver (and/or hepatic system), kidney (and/or renal system), brain (and/or cerebral system) such as headaches including migraine headaches, pancreas (and/or pancreatic system), and lungs (and/or pulmonary system). “Disease” may include chronic conditions, for example, chronic conditions that result in the development of fibrosis. “Disease” also may include cancerous states, for example cancerous states that result in fibrotic tumors. “Injury” may include, for example, infarction, blunt trauma, and/or trauma experienced during or after surgery. See, e.g., Xu et al., “Protective effects of 5,4′-dihydroxy-3,5′-diemethoxy-7-O-beta-D-glucopyranosyloxy-flavone on experimental hepatic injury,” WORLD J. GASTROENTEROL. (2005) 11(12):1764-8; Gerrah et al., “Beneficial effect of aspirin on renal function in patients with renal insufficiency postcardiac surgery,” J. CARDIOVASC. SURG (TORINO) (2004) 45(6):545-50; and Meng et al., “Effect of resveratrol on microcirculation disorder and lung injury following severe acute pancreatitis in rats,” WORLD J. GASTROENTEROL. (2005) 11(3):433-35. For example, chronic diseases, cancerous states, injury, or post-surgical trauma may result in the development of fibrosis in a selected tissue and consequently reduced circulation. By reducing whole blood viscosity, circulation may be improved in these instances. The reduction in whole cell viscosity is a separate benefit from any fibrinolytic treatment that also may be administered to address fibrosis.

A “patient in need thereof” also may include a patient who has anemia and who has or who is at risk for developing elevated whole blood viscosity as a result of treatment for anemia. For example, a “patient in need thereof” may include a patient with anemia who is undergoing erythropoietin therapy, which, as an adverse side-effect, may result in elevated whole blood viscosity. See, e.g., Hassan et al., “Effect of erythropoietin therapy on red cells filterability and left ventricular mass in predialysis patients,” REN. FAIL. (2005) 27(2):177-82. In accordance with the methods described herein, this adverse side effect may be treated by administering a composition that includes nattokinase before, during, or after the erythropoietin therapy to reduce whole blood viscosity.

A “patient in need thereof” also may include a patient who has been exposed or who is at risk for exposure to heavy metals. A “patient in need thereof” may include a patient with heavy metal poisoning (e.g., lead poisoning). For example, exposure to lead may result in elevated whole blood viscosity. See, e.g., Toplan et al., “Changes in hemorheological parameters due to lead exposure in female rats,” J. TRACE ELEM. MED. BIOL. (2004) 18(2): 179-82. In accordance with the methods described herein, an increase in whole blood viscosity resulting from heavy metal exposure may be treated by administering a composition that includes nattokinase.

A “patient in need thereof” also may include a patient with osteoarthritis or at risk for developing osteoarthritis. For example, osteoarthritis and/or the pain associated with osteoarthritis may be acerbated by poor circulation, which may be improved by administering a treatment that reduces whole blood viscosity. Such a treatment is provided by the methods described herein.

A “patient in need thereof” also may include a patient who has or who is at risk for developing a specific disease and/or syndrome that is linked to and/or characterized by elevated whole blood viscosity. Such diseases and/or syndromes may include, but are not limited to, Behcet's disease, Chagas' disease, Cushing syndrome, and/or Waldenstrom's disease. See, e.g., Ricart et al., “Haemorheological alterations in Behcet's disease are not related to tendency for venous thrombosis,” THROMB. RES. (2005) 115(5):399-404; Berra et al., “Blood viscosity changes in experimentally Trypanosoma cruzi-infected rats,” CLIN. HEMORHEOL. MICROCIRC. (2005) 32(3):175-82; Windberger et al., “Hemorheology in spontaneous animal endocrinopathies,” CLIN. HEMORHEOL. MICROCIRC. (2005) 31(3):207-15; and Sweeting et al., “Waldenstrom's disease and cardiopulmonary bypass: a case report,” PERFUSION (2004) 19(6):381-3.

A “patient in need thereof” also may include a patient in need of therapy to prevent platelet aggregation. For such patients, nattokinase may be administered (before or after) or co-administered with anti-platelet or anti-coagulant agents.

A “patient in need thereof” also may include a patient with prosthetic valves. A “patient in need thereof” also may include a patient having or at risk for developing arrhythmias and thrombosis. In some embodiments, nattokinase and warfarin are administered to treat such patients. Nattokinase may be co-administered with warfarin or administered before or after warfarin.

A “patient in need thereof” also may include a patient who has or who is at risk for developing vascular diseases or conditions (e.g., diseases or conditions associated with abnormally slow blood circulation). For example, a “patient in need thereof” may include a patient who has or who is at risk for developing capillary-fragility, telangiectasia, phlebostasis or venostasis and associated conditions such as “spider veins” and “varicose veins.” As such, the present compositions may be used by a patient cosmetically to improve the appearance of skin by inhibiting the formation of unsightly skin conditions such as spider veins and/or varicose veins. For such patients, the present composition may be administered in oral form or other form, including topical form.

A “patient in need thereof” also may include a patient who is desirous of other cosmetic effects observed by administration of the present nattokinase compositions. For example, a “patient in need thereof” may include a patient who is desirous of enhanced nail growth

Patients with these types of diseases and syndromes may benefit from a treatment regimen that results in a reduction in whole blood viscosity, and therefore may benefit from the methods described herein.

“Nattokinase”

As used herein, “nattokinase” refers to the enzyme known in the art (Enzyme Commission Number EC 3.4.21.62 and CAS Registry Number 9014-01-1) for its pro-fibrinolytic activity. “Nattokinase” is present in the vegetable cheese-like food called Natto, and can be administered as such. Alternatively, nattokinase may be extracted and purified from Natto, as described in more detail below.

“Nattokinase” also includes a natural, synthetic or recombinantly produced polypeptide comprising an amino acid sequence of SEQ ID NO:2 (i.e., nattokinase precursor) and/or SEQ ID NO:3 (i.e., processed nattokinase), and includes a polypeptide consisting of an amino acid sequence of SEQ ID NO:2 and/or SEQ ID NO:3.

“Nattokinase” also includes polypeptides that are fragments or variants of the amino acid sequences of SEQ ID NO:2 and/or SEQ ID NO:3. For example, a nattokinase fragment may include a polypeptide having an amino acid sequence of at least about 200 contiguous amino acids of SEQ ID NO:3. Nattokinase variants include polypeptides having at least about 75% sequence identity, at least about 80% sequence identity, or at least about 90% sequence identity to SEQ ID NO:3. Nattokinase variants preferably have nattokinase activity (e.g., fibrinolytic activity). Nattokinase variants may have at least about 30%, 50%, or 70% of the wild-type level of nattokinase activity (fibrin units/mole).

Additionally, nattokinase variants include polypeptides having an amino acid sequence that is at least about 95% and/or at least about 99% identical to SEQ ID NO:3. These nattokinase variants may include conservative amino acid substitutions. Examples of conservative substitutions include substitutions by an amino acid of a similar polarity and/or substitutions by an amino acid from the same class of amino acids. For example, nonpolar (hydrophobic) amino acids include alanine, leucine, isoleucine, valine, proline, phenylalanine, tryptophan and methionine. Polar neutral amino acids include glycine, serine, threonine, cysteine, tyrosine, asparagine, and glutamine. Positively charged (basic) amino acids include arginine, lysine and histidine. Negatively charged (acidic) amino acids include aspartic acid and glutamic acid. Nattokinase variants also may have one or more amino acid additions or deletions relative to SEQ ID NO:2 and/or SEQ ID NO:3. For example, the polypeptides may have up to about 5, up to about 10, up to about 15 or up to about 20 amino acid additions or deletions (e.g., at the N-terminus, internal, or at the C-terminus).

Those skilled in the readily can make the above described nattokinase variants by methods that are routine in the art. The nattokinase variants can be tested for nattokinase activity using methods known in the art (e.g., using fibrinolytic assays). The nattokinase variants can be screened for suitability for use in the present invention by assessing their ability to reduce whole cell viscosity using methods known in the art and described below.

Methods of Preparing Nattokinase

Nattokinase may be prepared by any suitable method. For example, nattokinase may be prepared by fermenting a suitable nutrient medium (e.g., a slurry of soybeans) with a suitable microorganism (e.g., a Bacillus strain such as Bacillus subtilis var. natto), and subsequently purifying nattokinase from the nutrient medium. Nattokinase may be purified from the fermented slurry by any suitable method such as centrifugation and/or filtration. Nattokinase may be prepared as a liquid solution and/or dried (e.g., spray dried onto a suitable neutral substrate) to provide a dry formulation. Nattokinase may also be prepared by any suitable recombinant method in which nattokinase is expressed and subsequently purified.

Methods for preparing nattokinase by suitable methods for suitable formulations have been described. See, e.g., U.S. Pat. No. 5,750,650; U.S. Pat. No. 6,730,504; U.S. Pat. No. 6,669,971; U.S. Pat. No. 6,537,543; U.S. Pat. No. 6,420,145; U.S. 2004-0043014; and U.S. 2004-0043015; all of which are incorporated by reference herein in their entireties.

Nattokinase may be extracted from the traditional Japanese food natto or from a pure culture of Bacillus subtilis var. natto. An aqueous extract of natto or a culture of Bacillus subtilis var. natto may be obtained by extracting with water or a neutral or weakly basic aqueous solution of salt(s). Aqueous solutions of salts may include, for example, a phosphate buffer (pH 6-8) that includes a salt such as 0.01-0.3M NaCl or KCl, or 0.005-0.1M tris(hydroxymethyl)aminomethane (“tris”) buffer (pH 7-9) that include 0.01-0.3M NaCl or KCl. Impurities in a crude fraction may be removed by adsorbing the fraction on an anion exchanger with a neutral or weakly basic buffer. Nattokinase may be purified by adsorption on a cation exchanger equilibrated with a neutral or weakly basic buffer solution followed by elution with a neutral or weakly basic buffer solution containing salt(s). Elution may be performed with a buffer solution such as 0.005-0.05M phosphate buffer (pH 6-8) that includes 0.2-1M (preferably 0.4-0.6M) NaCl, or 0.005-0.5M tris-buffer (pH 7-9). The enzyme may be purified further by gel filtration on a carrier equilibrated with a neutral or weakly basic buffer solution. The carrier may be equilibrated with a buffer such as 0.005-0.05M phosphate buffer solution (pH 6-8) that includes 0.05-0.5M (preferably 0.2M) NaCl, or 0.005-0.05M tris-buffer solution (pH 7-9).

In one embodiment, nattokinase may be prepared by the following steps:

1. Adding alcohol or ammonium sulfate to an aqueous extract of natto or pure culture of Bacillus subtilis var. natto to precipitate a crude fraction;

2. Applying the crude fraction to a column that includes a hydrophobic carrier equilibrated with a neutral or weakly basic buffer to adsorb the fraction, and eluting the adsorbed fraction from the column with water or neutral or weakly basic buffer;

3. Passing the eluate through an anion exchanger equilibrates with a neutral or weakly basic buffer to adsorb the impurities onto the exchanger, and obtaining a purified fraction as the effluent, (alternatively, applying the eluate on a cation exchanger equilibrated with a neutral or weakly basic buffer to adsorb the fraction onto the exchanger and eluting a purified fraction with a neutral or weakly basic buffer that includes a salt);

4. Applying the purified fraction on a gel filtration carrier equilibrated with a neutral or weakly basic buffer that includes a salt;

5. Recovering purified nattokinase;

6. Optionally, spray drying the recovered purified nattokinase using a food carrier such as dextrin to provide a substance suitable for inclusion in a variety of enteral or rectal delivery systems.

Nattokinase may also be prepared by using recombinant methods. For example, the gene for nattokinase (e.g., SEQ ID NO:1) may be cloned into a suitable expression vector. The expression vector may be used to transform cells which are capable of expressing nattokinase under suitable conditions (e.g., under fermenting conditions). The expressed nattokinase then may be purified and prepared by any suitable means as described above (e.g., centrifugation, filtration, and/or drying).

Dosages and Formulations

The method disclosed herein typically includes administering a composition that comprises nattokinase. The method may include administering nattokinase at a dosage of about 2,000 fibrin units per day. In some embodiments, the method may include administering nattokinase at a dosage of about 4,000 or 6,000 fibrin units per day. Nattokinase activity as reported in fibrin units, may be obtained by the spectrophotometric measurement of the amount of acid-soluble low molecular weight products, which are observed to increase in concentration as a result of nattokinase hydrolyzing specific peptide linkages in fibrin. A “fibrin unit” is defined as the amount of nattokinase that increases the absorbance of the filtrate (i.e., the amount of the low molecular weight products) at 275 nm by 0.01 per minute under specified conditions. The Japan Health Food Authorization has certified the fibrin unit as the official measurement of nattokinase.

A composition useful in the present invention may include a threshold level of about 5 mg of nattokinase per serving or dose. Alternatively, a composition may include nattokinase at a level of about 5-500 mg per serving or dose (including about 5-50 mg per serving or dose). In one embodiment, the composition includes nattokinase at a level of about 100 mg per serving or dose.

Nattokinase may be administered as part of a composition, which may be a medical food, a pharmaceutical composition, or a mixture thereof. Nattokinase may be administered in the form of a powder, capsule, tablet, caplet, liquid, soft chew, chewing gum, bar (i.e., food bar), sublingual drop formulation or any other suitable form, and may be administered alone or in combination with other ingredients, such as in a food or beverage.

Additional Therapeutic Agents

The present invention also encompasses administering a composition that includes therapeutic agents in addition to nattokinase. For example, the composition may include at least one additional therapeutic agent selected from the group consisting of an anti-coagulant, a lipid-lowering agent and/or a cholesterol-lowering agent, a protease, an angiotensin-converting enzyme inhibitor, a calcium channel blocker, a diuretic, an anti-oxidant, and combinations thereof. Alternatively, the at least one therapeutic agent may be administered prior to, concurrently with, or subsequently to the nattokinase. The composition may be formulated as part of a kit for reducing whole blood viscosity which may include instruction for reducing whole blood viscosity. Additional therapeutic agents may be present in the same composition or a separate composition in the kit.

In formulations of the compositions that include additional therapeutic agents, nattokinase may be formulated as an active ingredient together with the additional therapeutic agent. In some formulations, nattokinase may be used to potentiate the effect of the additional therapeutic agent and/or to facilitate administration of the additional therapeutic agent. For example, nattokinase may be used to reduce blood viscosity where reduced blood viscosity potentiates the effect of an additional therapeutic agent and/or facilitates administration of the additional therapeutic agent. In formulations of the compositions in which nattokinase is used to potentiate the effect of the additional therapeutic agent and/or to facilitate administration of the additional therapeutic agent, the additional therapeutic agent may be present at a lower dose relative to a composition that does not include nattokinase. In other embodiments, it may be desirable to administer a nattokinase composition before, concurrently, or after the additional therapeutic agent to potentiate the effect of the additional therapeutic agent and/or to facilitate administration of the additional therapeutic agent.

Additional therapeutic agents may include anti-coagulants, such as aspirin and/or Coumadin. Additional therapeutic agents also may include lipid lowering agents, such as statins and/or lipases. Additional therapeutic agents also may include proteases such as bromelain, papain, and/or lumbrokinase. Additional therapeutic agents also may include angiotensin-converting enzyme inhibitors, such as trandolapril. Additional therapeutic agents also may include calcium channel blockers, such as verapamil. Additional therapeutic agents also may include diuretics such as hydrochlorothiazide. Additional therapeutic agents may include nitrous oxide synthetase inhibitors, such as L-arginine. Additional therapeutic agents may include alpha blockers. Additional therapeutic agents may include antibiotics, such as antibiotics selected from the following groups of antibiotics: Ample Spectrum Penicillins, Penicillins and Beta Lactamase Inhibitors, Cephalosporins, Macrolides and Lincosamines, Quinolones and Fluoroquinolones, Carbepenems, Monobactams, Aminoglycosides, Glycopeptides, Tetracyclines, Sulfonamides, Rifampin, Oxazolidonones, Streptogramins.

Additional therapeutic agents may include vitamins, minerals, sugars, and mixtures thereof. For example, additional therapeutic agents may include a vitamin selected from the group consisting of folic acid, thiamin, riboflavin, niacin, vitamins B6 and B12, pantothenic acid, biotin, choline, and mixtures thereof. In one embodiment, additional therapeutic agents include L-methylfolate, pyridoxal 5′-phosphate (B6), and methylcobalamin (B12). Minerals may include magnesium, calcium, zinc, selenium, and mixtures thereof. Suitable sugars may include D-ribose. A suitable dose of D-ribose may be in the range of 500-2000 mg daily, which may be divided into two doses.

Additional therapeutic agents may include anti-oxidants. Suitable anti-oxidants may include thiotic acid, vitamins A, C, and or E, selenium, flavonoids (e.g., those present in ashitaba chalcone powder), and mixtures thereof. In some embodiments, the additional agent is ashitaba chalcone powder, administered at a dose of 100-2000 mg daily.

Additional therapeutic agents may also include amino acids. For example, suitable amino acids include L-arginine. A suitable dose of L-arginine may be 2000-3000 mg daily.

Additional therapeutic agents may include lipid-lowering agents and/or cholesterol-lowering agents. Suitable lipid-lowering agents may include nicotinic acid.

Other additional therapeutic agents may include flavonoids (e.g., flavone or the flavonoids present in ashitaba chalcone powder, hydroxyethylrutosides (HER), catechin, epicatechin, epicatechin gallate, epigallocatechin gallate, proanthocyanidins, hesperidin, quercetin, rutin (a sugar of quercetin), and tangeritin), banana juice, betaine (trimethyl glycin), black soybean, coconut milk, coenzyme Q₁₀, cyclodextrin, enzymatically-modified hesperidin, enzymatically-modified rutin, gingko leaf extract, grape seed oil, parsley seed oil, phosphatidylserine, purified fish oil (EPA oil), quercetin, red malt (extract), rice germ extract, including γ-aminobutyric acid (GABA), sodium ascorbate, soybean lecithin, theanine, turmeric, vinca minor extract, vitamin E (oil), and mixtures thereof.

Additional therapeutic agents may include agents capable of counteracting the effect of nattokinase and/or any other additional therapeutic agent. For example, additional therapeutic agents may include vitamin K, ascorbate, black sesame paste, garlic powder, grape seed extract, hawthorn (Crataegus cuneata) extract, polyphenol extracted from apple, vinegar (e.g., apple vinegar, Koji (black) vinegar), and mixtures thereof.

The composition may also include a carrier or excipient, which is intended to mean substances that are substantially harmless to the individual to which the composition will be administered. Such an excipient, if present, normally fulfills the requirements given by national drug agencies. Official pharmacopeias such as the U.S.A. Pharmacopeia, the British Pharmacopeia, and the European Pharmacopeia set standards for well-known pharmaceutically acceptable carriers and excipients.

Suitable carriers and excipients may include all kinds that may be used for solid, semi-solid, fluid, or other dosage units. Suitable carriers and excipients may include solvents, buffering agents, preservatives, humectants, chelating agents, antioxidants, stabilizers, emulsifying agents suspending agents, gel-forming agents, diluents, disintegrating agents, binding agents, lubricants, coating agents, and wetting agents. Typically, the diluents and disintegrating agents may be lactose, saccharose, calcium phosphatases, calcium carbonate, calcium sulfate, mannitol, starches, and cellulose.

Binding agents may include saccharose, sorbitol, gum acacia, sodium alginate, gelatin, starches, cellulose, sodium carboxymethylcellulose, methylcellulose, hydroxypropylcellulose, polyvinylpyrrolidone, and polyethyleneglycol.

In some embodiments, the composition includes one or more lipophilic or amphiphilic agents. For example, the composition may include fatty acids. Exemplary fatty acids may include fatty acids present in fish oil such as eicosapentaenoic acid (EPA) and docosahexaenoic acid (DHA). Suitable doses of fish oil include doses effective to maximize anti-platelet aggregation (e.g., 2-4 grams per day).

The one or more lipophilic or amphiphilic agents may form liposomes that encapsulate a nattokinase composition to provide a liposomal formulation. The liposomal formulation may be engineered to possess one or more desirable characteristics such as increased absorption of nattokinase, decreased time of absorption of nattokinase (e.g., in vivo fibrinolytic activity achieved in less than 1-2 hours), observed in vivo fibrinolytic activity per oral administration, and controlled and measured release of nattokinase for daily oral dosing.

The composition disclosed herein may be formulated for oral, topical, transdermal, subcutaneous, parenteral, or pulmonary (e.g., aerosolized) administration. Oral, formulations are preferable. Oral and/or other formulations may include tablets, capsules, granules, powders, suspensions, liquids, and/or emulsions. Transdermal formulations may include patches or pads.

For topical formulations, nattokinase may be formulated as an exfoliant or debriding composition. Topical compositions may include additional exfoliants and/or debriding agents (e.g., fruit acids and proteolytic enzymes).

It has been discovered that the lymphatic system absorbs nattokinase efficiently. In accordance with one embodiment, the composition is be formulated to further enhance lymphatic absorption. For example, a liposomal formulation may be engineered to achieve lymphathic delivery via subcutaneous injection, intramuscular injection, intraperitoneal injection, and/or oral delivery. The liposomal composition may have mucoadhesive properties. The liposomal formulation may be targeted to Peyer patches or engineered to avoid Peyer patches. The liposomal composition may include a liposomal adjuvant.

It also has been discovered that nattokinase can be administered via a naso-gastrointestinal tube (“NG tube”) or a percutaneous endoscopic gastrostomy tube (“PEG tube”). Thus, in one embodiment, the composition is formulated for delivery via an NG tube or a PEG tube. In some embodiments, formulations include capsules, vials, or aliquots that encase a liquid nattokinase composition.

The aforementioned compositions may be formulated together with a matrix that controls the release of nattokinase as an active ingredient (e.g., a matrix for slow release of nattokinase). The matrix typically is a solid formulation which allows for the controlled, prolonged, or extended release of nattokinase at a rate sufficient to maintain therapeutic blood levels of nattokinase over a period of time (e.g., 24-30 hours, 1-7 days, 1-30 days or longer). The matrix can represent from about 40% to about 98% of the total weight of a composition or a unit dosage form, typically excluding any coatings in the case of tablets. In some embodiments, the controlled release matrix will represent from about 50% to about 95% of the total weight of the compositions. The matrix to nattokinase ratio can be from about 5 to 1 to about 15 to 1, and compositions having integer ratios of all possible combinations between these ranges including 10 to 1 are considered embodiments of the present invention.

The matrix can be any suitable material that provides sustained, controlled, or slow release of nattokinase. Rate controlling materials which may be used in the present invention include both synthetic and naturally occurring gums and/or polymers and other art-known rate controlling substances. Non-limiting examples include naturally occurring or modified naturally occurring or synthetic or semi-synthetic polymers or gums such as, e.g., alginates, carrageenan, pectin, xanthan gum, locust bean gum, guar gum, modified starch, alkylcellulose, hydroxypropylmethylcellulose, methylcellulose, and other cellulosic materials or polymers, such as sodium carboxymethylcellulose and hydroxypropylcellulose and mixtures of the foregoing. Additional synthetic and/or semisynthetic polymers include, e.g., cellulose acetate phthalate (CAP), polyvinylacetate phthalate (PVAP), hydroxypropylmethylcellulose phthalate, and/or acrylic polymers, such as methacrylic acid ester copolymers, zein, and the like. The matrix can include ingredients such as polysaccharides, cationic crosslinking agents, inert diluents, alkalizing agents, surfactants, polar solvents and other excipients.

Hemorheological Effects

The method disclosed herein results in a significant reduction in a patient's whole blood viscosity. As used herein, “whole blood viscosity” is the inverse of whole blood fluidity. Whole blood viscosity refers to a property through which whole blood has resistance to flow or shear. Whole blood viscosity may be influenced by a number of factors including, but not limited to, hematocrit (i.e., the percent red blood cell packed volume in a sample of blood), plasma proteins, temperature, and shear rate. Whole blood is a non-Newtonian fluid in that the viscosity of whole blood varies inversely with respect to shear rate.

Whole blood viscosity may be determined using any suitable method and/or instrument for measuring whole blood viscosity. For example, whole blood viscosity (“WBV”) may be determined using the following equation, WBV=S_{S}/S_{R}, where S_{S} is “Shear Stress”; and S_{R} is “Shear Rate” at a particular rate of shear. See, e.g., Hussain et al., “Relationship between power law coefficients and major blood constituents affect the whole blood viscosity,” J. BIOSCI. (India) (September 1999) 24(3):329-37. Whole blood viscosity may also be defined by the coefficients “n” and “k” as used in the equation: WBV=kS_{S}/S_{R}⁽ⁿ⁻¹⁾. In this equation, “n” a non-Newtonian behavior index value and “k” is a flow consistency index value.

A patient's whole blood viscosity may be measured by using any suitable instrument. For example, whole blood viscosity may be measured using a viscometer (e.g., an oscillating or capillary viscometer). Suitable instruments include a Rheolog® brand scanning capillary rheometer (Rheologics, Exton, Pa., USA). See also U.S. Pat. No. 6,019,735; U.S. Pat. No. 6,261,244; U.S. Pat. No. 6,152,888; U.S. Pat. No. 6,659,965; U.S. Pat. No. 6,152,888; U.S. Pat. No. 6,077,234; U.S. Pat. No. 6,193,667; U.S. Pat. No. 6,200,277; U.S. Pat. No. 6,322,524; U.S. Pat. No. 6,402,703; U.S. Pat. No. 6,428,488; U.S. Pat. No. 6,624,435; U.S. Pat. Nos. 6,322,525; 6,484,566; U.S. Pat. No. 6,484,565; U.S. Pat. No. 6,412,336; U.S. Pat. No. 6,450,974; U.S. Pat. No. 6,497,669; U.S. Pat. No. 6,598,465; U.S. Pat. No. 6,571,608; U.S. Pat. No. 6,523,396; and U.S. Pat. No. 6,564,618, the contents of which are incorporated by reference herein in their entireties.

Typically, the patient's whole blood viscosity will be determined at a hematocrit of about 30-50%, optionally at about 40%. Also, typically, a patient's whole blood viscosity will be measured through a range of shear rates. A typical shear rate range may include 1-1000 s⁻¹ or 1-100 s⁻¹.

A “significant reduction” in whole cell viscosity means that the patient's whole blood viscosity is reduced an average of at least about 5% over a shear rate range of 1-1000 s⁻¹ at a hematocrit of about 30-50%. In exemplary embodiments, the method of the present invention achieves a whole blood viscosity reduction of an average of at least about 10%, 15%, 20%, 25%, or 30% over a shear rate range of 1-1000 s⁻¹ at a hematocrit of about 30-50%.

In one embodiment, the method disclosed herein results in a reduction in red blood cell aggregation. Red blood cell aggregation may be measured and determined by any suitable method. See, e.g., Marton et al., “Red Blood Cell Aggregation Measurements in Whole Blood and in Fibrinogen Solution by Different Methods,” CLIN. HEMORHEOL. (2001) 24(2):75-83. Suitable instruments for measuring aggregation include an aggregometer (e.g., Myrenne MA-1 brand aggregometer, Myrenne GmbH).

In another embodiment, the method disclosed herein results in an increase in red blood cell deformability and/or perfusion (and/or a reduction in red blood cell rigidity). Suitable instruments for measuring deformability/rigidity include a diffractometer (e.g., Myrenne Rheodyn brand laser diffractometer, Myrenne GmbH).

The following examples are provided for illustrative purposes only and should not be construed as limiting the scope of the claims.

### EXAMPLE 1

Blood samples were obtained from healthy individuals. See Pais, et al., “Effects of nattokinase, a pro-fibrinolytic enzyme, on red blood cell aggregation and whole blood viscosity,” CLIN. HEMORHEOLOGY AND MICROCIRCULATION (2006) 00:1-4 (incorporated by reference herein in its entirety). The samples were placed into an EDTA solution and were incubated with Nattokinase at concentrations of 15.60 μM, 31.25 μM, 62.50 μM, and 125 μM for 30 minutes at 37° C. The hematocrit for the samples was adjusted to 40% and red blood cell aggregation was measured using a Myrenne MA-1 aggregometer (Myrenne GmbH). Whole blood viscosity was assessed with a computer controlled scanning capillary Rheolog® rheometer (Rheologics, Exton, Pa. USA) over a shear rate range of 1-1000 s⁻¹.

We observed a significant and dose-dependent decrease in red blood cell aggregation in the presence of Nattokinase. We also observed a significant decrease in whole blood viscosity.

### EXAMPLE 2

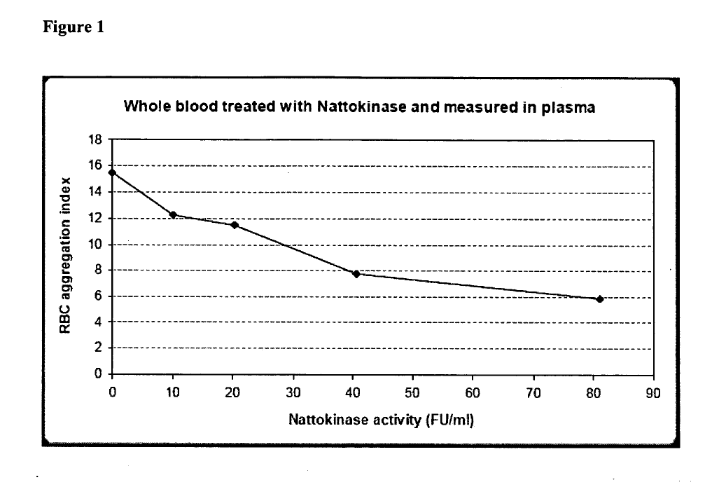

Venous blood was collected an placed into an EDTA solution. Cell suspensions were prepared that included whole blood or washed red blood cells in PBS. Nattokinase was added to achieve various enzyme activities: 10.125 FU/ml; 20.25 FU/ml; 40.5 FU/ml; or 81 FU/ml. The samples were incubated for 30 minutes at 37° C. For the samples including red blood cells in PBS, the red blood cells were resuspended in dextran 70 solution (70 kDa, 3 g/dL). Red blood cell aggregation was measured at statis in a Myrenne Aggregometer (Myrenne GmbH). Results are provided for whole blood in FIG. 1, which shows a dose-dependent decrease in red blood cell aggregation in the presence of Nattokinase.

### EXAMPLE 3

Venous blood was collected and placed into an EDTA solution. Nattokinase was added to achieve various enzyme activities: 37.5 FU/ml, 75 FU/ml, or 150 FU/ml. The samples were incubated for 30 minutes at 37° C. The red blood cells present in the samples were washed twice and then re-suspended in untreated plasma or in dextran 70 solution (70 kDa, 3 g/dL). Red blood cell aggregation was measure at statis in a Myrenne Aggregometer (Myrenne GmbH).

All references, patents, and/or applications cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compounds/compositions described herein as presently representative of preferred embodiments are exemplary and are not intended as limitations on the scope of the invention. Changes therein and other uses will occur to those skilled in the art, which are encompassed within the invention.

It will be readily apparent to one skilled in the art that varying substitutions and modifications may be made to the invention disclosed herein without departing from the scope and spirit of the invention. For example, a variety of different binding pairs can be utilized, as well as a variety of different therapeutic and diagnostic agents. Thus, such additional embodiments are within the scope of the present invention.

The invention illustratively described herein may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

1. A method for reducing whole blood viscosity in a patient in need thereof, comprising administering to the patient a composition that comprises nattokinase.

2. The method of claim 1, wherein the patient has or is at risk for a vascular disease and/or injury.

3. The method of claim 2, wherein the vascular disease and/or injury is selected from the group consisting of cerebral vascular disease and/or injury and cardiovascular disease and/or injury.

4. The method of claim 1, wherein the patient has essential hypertension.

5. The method of claim 4, wherein the patient has a systolic blood pressure of at least about 170 mm Hg and/or a diastolic blood pressure of at least about 100 mm Hg.

6. The method of claim 1, wherein the patient is diabetic.

7. The method of claim 1, wherein the patient is pregnant and has or is at risk for intrauterine growth restriction and/or pre-eclampsia.

8. The method of claim 1, wherein the patient has a disease or injury selected from the group consisting of hepatic disease and/or injury, renal disease and/or injury, cerebral disease and/or injury, pancreatic disease and/or injury, pulmonary disease and/or injury, and combinations thereof

9. The method of claim 1, wherein the patient has anemia and is undergoing erythropoietin therapy.

10. The method of claim 9, wherein the patient has heavy metal poisoning.

11. The method of claim 1, wherein the patient has a disease or syndrome selected from the group consisting of Behcet's disease, Chagas' disease, Cushing syndrome, Waldenstrom's disease.

12. The method of claim 1, wherein the patient's whole blood viscosity is reduced an average of at least about 5% over a shear rate range of 1-1000 s⁻¹ at a hematocrit of about 30-50%.

13. The method of claim 1, wherein red blood cell aggregation is reduced.

14. The method of claim 1, wherein red blood cell deformability is increased.

15. The method of claim 1, wherein the nattokinase is administered at a dosage of at least about 2,000 fibrin units per day.

16. The method of claim 1, wherein the nattokinase comprises a polypeptide having at least about 90% sequence identity to SEQ ID NO:3.

17. The method of claim 1, wherein the composition is administered via a naso-gastrointestinal tube or a percutaneous endoscopic gastrostomy tube.

18. The method of claim 1, wherein the nattokinase is prepared from an extract of soy beans that have been fermented with Bacillus subtilis var. natto.

19. A method for reducing whole blood viscosity in a patient in need thereof, comprising administering to the patient a composition that comprises nattokinase and at least one additional therapeutic agent selected from the group consisting of an anti-coagulant, a lipid lowering agent, a protease, an angiotensin-converting enzyme inhibitor, a calcium channel blocker, a diuretic, an anti-oxidant, a nitrous oxide synthetase inhibitor, an alpha blocker, and combinations thereof.

20. A method for enhancing therapeutic effect of a therapeutic agent comprising administering an effective dose of nattokinase before, concurrently, or after the therapeutic agent.


SEQUENCE LISTING (3 sequences)

SEQ ID NO: 1 - DNA, 1146 bp, Bacillus subtilis
gtgagaagcaaaaaattgtggatcagcttgttgtttgcgttaacgttaatctttacgatggcgttcagcaacatgtctgcgcaggctgccggaaaaagcagtacagaaaagaaatacattgtcggatttaagcagacaatgagtgccatgagttccgccaagaaaaaggatgttatttctgaaaaaggcggaaaggttcaaaagcaatttaagtatgttaacgcggccgcagcaacattggatgaaaaagctgtaaaagaattgaaaaaagatccgagcgttgcatatgtggaagaagatcatattgcacatgaatatgcgcaatctgttccttatggcatttctcaaattaaagcgccggctcttcactctcaaggctacacaggctctaacgtaaaagtagctgttatcgacagcggaattgactcttctcatcctgacttaaacgtcagaggcggagcaagcttcgttccttctgaaacaaacccataccaggacggcagttctcacggtacgcatgtcgccggtacgattgccgctcttaataactcaatcggtgttctgggcgtagcgccaagcgcatcattatatgcagtaaaagtgcttgattcaacaggaagcggccaatatagctggattattaacggcattgagtgggccatttccaacaatatggatgttatcaacatgagccttggcggacctactggttctacagcgctgaaaacagtagttgataaagcggtttccagcggtatcgtcgttgctgccgcagccggaaacgaaggttcatccggaagcacaagcacagtcggctaccctgcaaaatatccttctactattgcagtaggtgcggtaaacagcagcaaccaaagagcttcattctccagcgtaggttctgagcttgatgtaatggctcctggcgtgtccatccaaagcacacttcctggaggcacttacggcgcttataacggaacgtccatggcgactcctcacgttgccggagcagcagcgctaattctttctaagcacccgacttggacaaacgcgcaagtccgtgatcgtttagaaagcactgcaacataccttggaagctctttctactatggaaaagggttaatcaacgtacaagcagctgcacaataa

SEQ ID NO: 2 - protein, 381 aa, Bacillus subtilis
MRSKKLWISLLFALTLIFTMAFSNMSAQAAGKSSTEKKYIVGFKQTMSAMSSAKKKDVISEKGGKVQKQFKYVNAAAATLDEKAVKELKKDPSVAYVEEDHIAHEYAQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSVGSELDVMAPGVSIQSTLPGGTYGAYNGTSMATPHVAGAAALILSKHPTWTNAQVRDRLESTATYLGSSFYYGKGLINVQAAAQ

SEQ ID NO: 3 - protein, 275 aa, Bacillus subtilis
AQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSVGSELDVMAPGVSIQSTLPGGTYGAYNGTSMATPHVAGAAALILSKHPTWTNAQVRDRLESTATYLGSSFYYGKGLINVQAAAQ